(19) United States
(12) Patent Application Publication
(10) Pub. No.: US 2007/0282007 A1
(43) Pub. Date: Dec. 6, 2007

(54) TREATMENT OF PAIN DISORDERS WITH trans 4-(3,4-DICHLOROPHENYL)-1,2,3,4-TETRAHYDRO-1-NAPHTHALENAMINE AND ITS FORMAMIDE

(75) Inventors: Paul Michael Tarantino, Holden, MA (US); Una Campbell, Marlborough, MA (US); Larry Bush, Worcester, MA (US)

Correspondence Address: HESLIN ROTHENBERG FARLEY & MESITI PC, 5 COLUMBIA CIRCLE, ALBANY, NY, 12203, US

(73) Assignee: SEPRACOR INC., Marlborough, MA (US)

(21) Appl. No.: 11/693,844

(22) Filed: Mar. 30, 2007

Related U.S. Application Data
(60) Provisional application No. 60/809,649, filed on May 31, 2006.

(51) Int. Cl. A61K 31/135; A61K 31/165; A61P 25/00; A61P 25/02

(52) U.S. Cl. 514/630; 514/647

(57) ABSTRACT

Treatment of pain disorders with (1R,4S)-trans 4-(3,4-dichlorophenyl)-1,2,3,4-tetrahydro-1-napthalenamine; and (1S,4R)-trans 4-(3,4-dichlorophenyl)-1,2,3,4-tetrahydro-1-napthalenamine is disclosed. A process for preparing 4-(3,4-dichlorophenyl)-1,2,3,4-tetrahydro-1-napthalenamine is also disclosed. The process includes the preparation of all four isomers of N-[4-(3,4-dichlorophenyl)-1,2,3,4-tetrahydronaphthalen-1-yl]formamide, which are also useful in treatment of pain disorders.

### CROSS REFERENCE TO RELATED APPLICATIONS

This application claims priority from U.S. provisional application 60/809,649, filed May 31, 2006, the entire disclosure of which is incorporated herein by reference.

### FIELD OF THE INVENTION

The present invention relates to methods of treating pain.

### BACKGROUND OF THE INVENTION

Clinicians recognize a distinction among different types of pain, such as nociceptive pain, psychogenic pain, and neuropathic pain. Nociceptive pain is a normal response caused by an acute injury to body tissues, such as extreme heat (or cold), pressure or incision (e.g. pinprick). Psychogenic pain is entirely or mostly related to a psychological disorder. Neuropathic pain can be a chronic disorder with no particular stimulus, or it can arise from more slowly developing responses to injury to nerves in the periphery, spinal cord, or brain. Some forms of neuropathic pain, such as allodynia, involve a perception of pain in response to normally non-noxious stimuli.

Neuropathic pain disorders constitute a large, varied group of chronic, painful disorders, varying in severity from moderate to severe, and are believed to involve neuronal hyperexcitability arising from some type of nerve damage. Neuropathic pain may be felt as a burning or tingling sensation or as hypersensitivity to touch or cold. Neuropathic pain includes such syndromes as phantom limb pain, postherpetic neuralgia, reflex sympathetic dystrophy, diabetic peripheral neuropathy, and causalgia.

As discussed above, one neuropathic pain disorder is phantom limb pain. Phantom limb pain is a pain that is seemingly felt in an amputated part of the body, usually a limb. It differs from phantom limb sensation—the feeling that the amputated part is still there—which is much more common. Phantom limb pain cannot be caused by a problem in the limb; rather, it must be caused by a change in the nervous system above the site where the limb was amputated. The brain misinterprets the nerve signals as coming from the amputated limb. Usually, the pain is perceived as arising from the toes, ankle, and foot of an amputated leg or the fingers and hand of an amputated arm. The pain may resemble squeezing, burning, or crushing sensations, but it often differs from any sensation previously experienced. For some people, phantom limb pain occurs less frequently as time passes, but for others, it persists.

Another neuropathic pain disorder is postherpetic neuralgia, which results from herpes zoster (“shingles”), which causes inflammation of nerve tissue. The pain is felt as a constant deep aching or burning, as a sharp and intermittent pain, or as hypersensitivity to touch or cold. The pain may be debilitating.

Some other types of neuropathic pain disorders are reflex sympathetic dystrophy (complex regional pain syndrome, type 1) and causalgia (complex regional pain syndrome, type 2). The reflex sympathetic dystrophy and causalgia are chronic pain syndromes. They are defined as persistent burning pain accompanied by certain abnormalities that occur in the same area as the pain. Abnormalities include increased or decreased sweating, swelling, changes in skin color, and damage to the skin, hair, nails, muscle, and bone (including muscle wasting and bone loss). Both syndromes typically occur after an injury. Reflex sympathetic dystrophy results from injury to tissues other than nerve tissue (as in the shoulder-hand syndrome). Causalgia results from injury to nerve tissue. Some types of reflex sympathetic dystrophy and causalgia are made worse by activity of the sympathetic nervous system, which normally prepares the body for stressful or emergency situations—for fight or flight.

Diabetic peripheral neuropathy is a common complication of diabetes mellitus. Diabetic peripheral neuropathy can manifest in a variety of ways. Some diabetes patients experience painful diabetic neuropathy (PDN), while others experience an asymptomatic, progressive loss of peripheral nerve function. Diabetic peripheral neuropathy can affect both the autonomic and the somatic nervous systems. Autonomic symptoms can include sexual dysfunction, bladder abnormalities, gastroparesis, orthostatic hypotension, and diabetic diarrhea. However, diabetic peripheral neuropathy most commonly affects the somatic, or sensorimotor, system. The sensorimotor system commonly exhibits peripheral symptoms in a distal symmetric pattern. These symptoms can be very painful and even disabling. They commonly present as burning, shooting, tingling, and allodynia. The diabetes patient may describe the symptoms as “a pain in the bones,” “walking on broken stones,” “a toothache in the feet,” or “feet on fire.” Patients may also display muscle weakness, incoordination, and ataxia from their nerve disorder. Diabetic peripheral neuropathy presents as a “glove and stocking” distribution, with symptoms usually beginning in the lower extremities, first affecting the toes and then progressing upward. Involvement of the upper extremities starts distally with the fingertips, then subsequently moves proximally up the hands and arms. Patients may lose their ability to detect pain and temperature sensations or may complain of paresthesias or dysesthesias. Loss of sensation predisposes the patient to the development of diabetic foot ulcers and infection. Resulting infections may lead to serious sequalae of cellulitis, osteomyelitis, or gangrene, with amputation as the only possible cure in some instances.

Monoamine reuptake inhibitors have shown some efficacy in the treatment of various neuropathic pain disorders. Tricyclic antidepressants that have been found to be most effective (e.g., imipramine and amitriptyline) posses both serotonin (5-hydroxytryptamine; 5-HT) and norepinephrine (NE) reuptake inhibitory properties. Tramadol, a mixed opioid/monoamine uptake inhibitor, also inhibits 5-HT and NE reuptake. Bupropion, a dual NE and dopamine (DA) reuptake inhibitor, has been shown to be efficacious in the treatment of neuropathic pain (Semenchuk et al., Neurology, 13:57(9): 1583-1588 (2001)). Another prior art approach for neuropathic pain of any type includes use of a selective 5-HT reuptake inhibitor (SSRI; e.g., fluoxetine, paroxetine).

However, there are toxicities associated with currently available therapies that could potentially be addressed by a more potent and selective drug. For example, greater than 25% of patients treated for up to 90 days with tramadol have experienced dizziness/vertigo, dysphoria, nausea, constipation or somnolence (ULTRAM®, Current Product Label). Clonidine causes a decrease in cardiac output and has been associated with drawal symptoms of rebound hypertension (CATAPRES® and CLORPRES®, Current Product Labels). Bupropion is associated with a dose-dependent risk of seizures (WELLBUTRIN®, Current Product Label). Gabapentin is thought to have tumorigenic potential and has been associated with a number of neuropsychiatric adverse events (NEURONTIN®, Current Product Label). The tricyclic antidepressants (including amitriptyline and imipramine) have been associated with seizures, sedation, hypotension and cardiac effects (most notably arrhythmias) (Goodman and Gilman, The Pharmacological Basis of Therapeutics, 10^{th} Ed. 2001). Moreover, none of the prior art monoamine reuptake inhibitor drugs for treatment of neuropathic pain disorders are capable of inhibiting the uptake of more than two monoamines at the same time.

Another pain disorder is fibromyalgia. The term “fibromyalgia” describes several disorders, all characterized by achy pain and stiffness in soft tissues, including muscles, tendons, and ligaments. Various alternative terms for fibromyalgia disorders have been used in the past, including generalized fibromyalgia, primary fibromyalgia syndrome, secondary fibromyalgia syndrome, localized fibromyalgia, and myofascial pain syndrome. Previously, these disorders were collectively called fibrositis or fibromyositis syndromes.

In generalized fibromyalgia, which is about seven times more common in women than in men, the pain and stiffness are widespread, occurring throughout the body. Primary fibromyalgia syndrome is the most common variation of generalized fibromyalgia; it usually occurs in young or middle-aged women who have no associated or contributing underlying disorder.

Secondary fibromyalgia syndrome is a type of generalized fibromyalgia and refers to fibromyalgia symptoms in a person who has another underlying disorder that is causing the fibromyalgia symptoms, such as hypothyroidism. Other disorders, such as systemic lupus erythematosus or rheumatoid arthritis, may be associated with fibromyalgia, but may not be the underlying cause.

In localized fibromyalgia, pain and stiffness occur in a particular area, or at a few sites, such as the jaw, neck, and/or shoulder muscles. Localized fibromyalgia is somewhat more likely to occur in men, possibly because they are more likely to engage in more physically strenuous activities in occupational or sports situations. Sometimes, localized fibromyalgia gradually spreads to become generalized fibromyalgia. Myofascial pain syndrome is a type of localized or regional fibromyalgia which may occur in various sites. In the temporomandibular type, the chewing muscles on the side of the face are commonly involved and may become painful and tender.

Usually, the cause of generalized fibromyalgia is unknown; in primary fibromyalgia syndrome, the cause is always unknown. However, generalized fibromyalgia may be worsened by physical or mental stress, poor sleep, repetitive strains, an injury, or chronic exposure to dampness and cold. In secondary fibromyalgia syndrome, an underlying cause is known. The syndrome may occur as a complication of certain infections (for example, Lyme disease), or hypothyroidism. Another associated disorder, such as rheumatoid arthritis or systemic lupus erythematosus, may be coincidental or may sometimes increase the symptoms of fibromyalgia.

Localized fibromyalgia often results from an occupational or recreational muscle strain. The temporomandibular type of myofascial pain syndrome can be caused by clenching and grinding of the teeth, especially while the person is asleep.

Aching stiffness and pain usually develop gradually in generalized fibromyalgia. In localized fibromyalgia, however, the pain may begin suddenly after muscle strains, and be sharp. In both syndromes, the pain usually worsens with fatigue, straining, or overuse. Specific discrete areas of muscle may be tender when firm fingertip pressure is applied, these areas are called either tender or trigger points (both points are tender, but “trigger” points radiate the pain to a distant site). During flare-ups, muscle tightness or even spasms may occur. Any soft tissue (muscles, tendons, or ligaments) may be affected. Soft tissue of the neck, shoulders, chest and rib cage, lower back, and thighs as well as joints are especially likely to be painful.

In primary fibromyalgia syndrome, widespread pain typically occurs and is often accompanied by other symptoms, such as poor sleep, anxiety, depression, fatigue, and irritable bowel syndrome.

In the temporomandibular type of myofascial pain syndrome, the mouth often cannot be opened fully, and opening the mouth may be painful. Clenching or grinding of the teeth during sleep can lead to a headache on awakening that improves over the course of the day. Sometimes the teeth clenching or grinding continues throughout the day.

Sertraline, whose chemical name (1S,4S)-cis 4-(3,4-dichlorophenyl)-1,2,3,4-tetrahydro-N-methyl-1-napthalenamine, is approved for the treatment of depression by the United States Food and Drug Administration, and is available under the trade name ZOLOFT® (Pfizer Inc., NY, N.Y., USA). In humans, sertraline has been shown to be metabolized to (1S,4S)-cis 4-(3,4-dichlorophenyl)-1,2,3,4-tetrahydro-1-napthalenamine, also known as desmethylsertraline or norsertraline. Desmethylsertraline has been described as “not contributing significantly to the serotonergic action of sertraline” Ronfield et al., Clinical Pharmacokinetics, 32:22-30 (1997). Reports from Hamelin et al., Clinical Pharmacology & Therapeutics, 60:512 (1996) and Serebruany et al., Pharmacological Research, 43:453-461 (2001), have stated that desmethylsertraline is “neurologically inactive”. These statements appear to be based on results observed in 5-HT-induced syndrome and ptosis in mouse models in vivo, whereas the original Pfizer research papers suggested on the basis of data in vitro that desmethylsertraline was a selective 5-HT uptake inhibitor. Koe et al., JPET, 226:686-700 (1983). Sanchez et al., Cellular and Molecular Neurobiology, 19: 467 (1999), speculated that despite its lower potency, desmethylsertraline might play a role in the therapeutic effects of sertraline but, there is presently no evidence in the literature to support this theory.

The primary clinical use of sertraline is in the treatment of depression. In addition U.S. Pat. No. 4,981,870 discloses and claims the use of sertraline and norsertraline, as well as (1R,4S)-trans 4-(3,4-dichlorophenyl)-1,2,3,4-tetrahydro-N-methyl-1-napthalenamine and (1S,4R)-trans 4-(3,4-dichlorophenyl)-1,2,3,4-tetrahydro-N-methyl-1-napthalenamine for the treatment of psychosis, psoriasis, rheumatoid arthritis and inflammation. The receptor pharmacology of the individual (1S,4R) and (1R,4S) enantiomers of trans 4-(3,4-dichlorophenyl)-1,2,3,4-tetrahydro-N-methyl-1-napthalenamine is described by Welch et al., J. Med. Chem., 27:1508-1515 (1984).

Furthermore, methods of treating central nervous system disorders using (1R,4S)-trans 4-(3,4-dichlorophenyl)-1,2,3,4-tetrahydro-1-napthalenamine; (1S,4R)-trans 4-(3,4-dichlorophenyl)-1,2,3,4-tetrahydro-1-napthalenamine and the four isomers of N-[4-(3,4-dichlorophenyl)-1,2,3,4-tetrahydronaphthalen-1-yl]formamide (an intermediate that may be used in the synthesis of norsertraline) are disclosed in US Pub. No. 2004-0092605 and WO 04/024669.

### SUMMARY OF THE INVENTION

The present invention relates to methods of using (1R,4S)-trans 4-(3,4-dichlorophenyl)-1,2,3,4-tetrahydro-1-napthalenamine (P) and (1S,4R)-trans 4-(3,4-dichlorophenyl)-1,2,3,4-tetrahydro-1-napthalenamine (Q) in the treatment of pain disorders, such as neuropathic pain disorders and fibromyalgia. In one aspect, the methods according to the present invention can produce diminished side effects as compared to current standards of treatment.

Compounds P and Q are represented by the formulae:

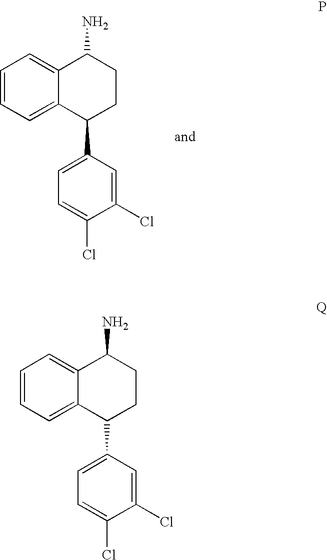

In one aspect, the present invention relates to a method for treating pain disorders, which involves the administration of a therapeutically effective amount of P or Q, or a pharmaceutically acceptable salt of either.

In another aspect, the invention relates to trans-4-(3,4-dichlorophenyl)-1,2,3,4-tetrahydro-1-napthalenamine of the formula (PQ):

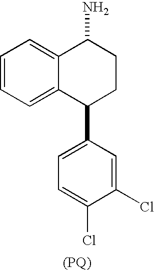

### DETAILED DESCRIPTION OF THE INVENTION

The present invention provides several embodiments of a method for treating pain disorders, such as neuropathic pain disorders and fibromyalgia. The method encompasses administering enantiomerically enriched P or enantiomerically enriched Q, or any mixture or pharmaceutically acceptable salt thereof. Thus, in one embodiment, the present invention is a method for treating a pain disorder in a human, the method comprising administering to a person in need of therapy for a pain disorder a therapeutically effective amount of a compound chosen from (1R,4S)-4-(3,4-dichlorophenyl)-1,2,3,4-tetrahydro-1-naphthalenamine (P); (1S,4R)-4-(3,4-dichlorophenyl)-1,2,3,4-tetrahydro-1-naphthalenamine (Q); mixtures of P and Q; and pharmaceutically acceptable salts thereof.

When the method encompasses administering a therapeutic amount of either enantiomerically enriched P or enantiomerically enriched Q, or pharmaceutically acceptable salt thereof, the term “enantiomerically enriched” refers to about 80% to about 100% enantiomeric excess of P or Q, respectively. Thus, the present invention is also a method for treating a pain disorder in a human, the method comprising administering to a person in need of therapy for a pain disorder a therapeutically effective amount of a compound chosen from (1R,4S)-4-(3,4-dichlorophenyl)-1,2,3,4-tetrahydro-1-naphthalenamine (P); (1S,4R)-4-(3,4-dichlorophenyl)-1,2,3,4-tetrahydro-1-naphthalenamine (Q); and pharmaceutically acceptable salts thereof; wherein P or Q is present in about 80% to about 100% enantiomeric excess. In one preferred embodiment, P or Q is present in about 90% to about 100% enantiomeric excess. In another preferred embodiment, P or Q is present in about 95% to about 100% enantiomeric excess. In yet another preferred embodiment, P or Q is present in about 99% to about 100% enantiomeric excess. The pain disorder may be neuropathic pain disorders or fibromyalgia.

The term “enantiomeric excess” is well known in the art and is defined for a resolution of ab into a+b as

$${ee}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee.; in other words, a 90% e.e. reflects the presence of 95% of one enantiomer and 5% of the other in the material in question.

The term “treating” when used in connection with one or more pain disorders means amelioration, prevention, or relief from one or more of the symptoms and/or effects associated with a pain disorder, and includes the prophylactic administration of P or Q, or a mixture thereof, or pharmaceutically acceptable salt thereof, to substantially diminish the likelihood or seriousness of the condition or disorder.

The term “pharmaceutically acceptable salt” refers to salts prepared from pharmaceutically acceptable non-toxic acids including inorganic acids and organic acids. Exemplary acids that form pharmaceutically acceptable salts with the amines of the invention and that may be used in the compositions of the present invention are acetic acid, benzenesulfonic (besylate) acid, benzoic acid, isethionic acid, camphorsulfonic acid, citric acid, ethenesulfonic acid, fumaric acid, gluconic acid, glutamic acid, hydrobromic acid, hydrochloric acid, lactic acid, maleic acid, malic acid, mandelic acid, methanesulfonic acid, mucic acid, nitric acid, pamoic acid, pantothenic acid, phosphoric acid, succinic acid, sulfuric acid, p-toluenesulfonic acid and tartaric acid. The hydrochloric acid salt is particularly preferred.

Preparation of compounds of the present invention is illustrated below in Scheme 1 and its accompanying narrative.

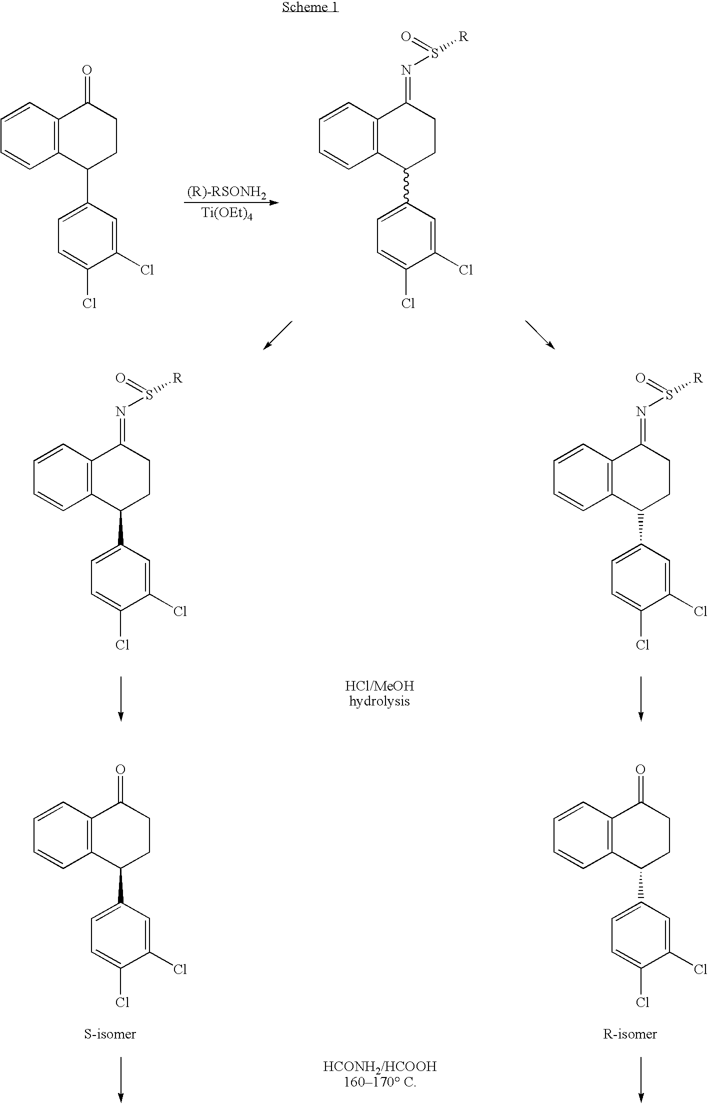

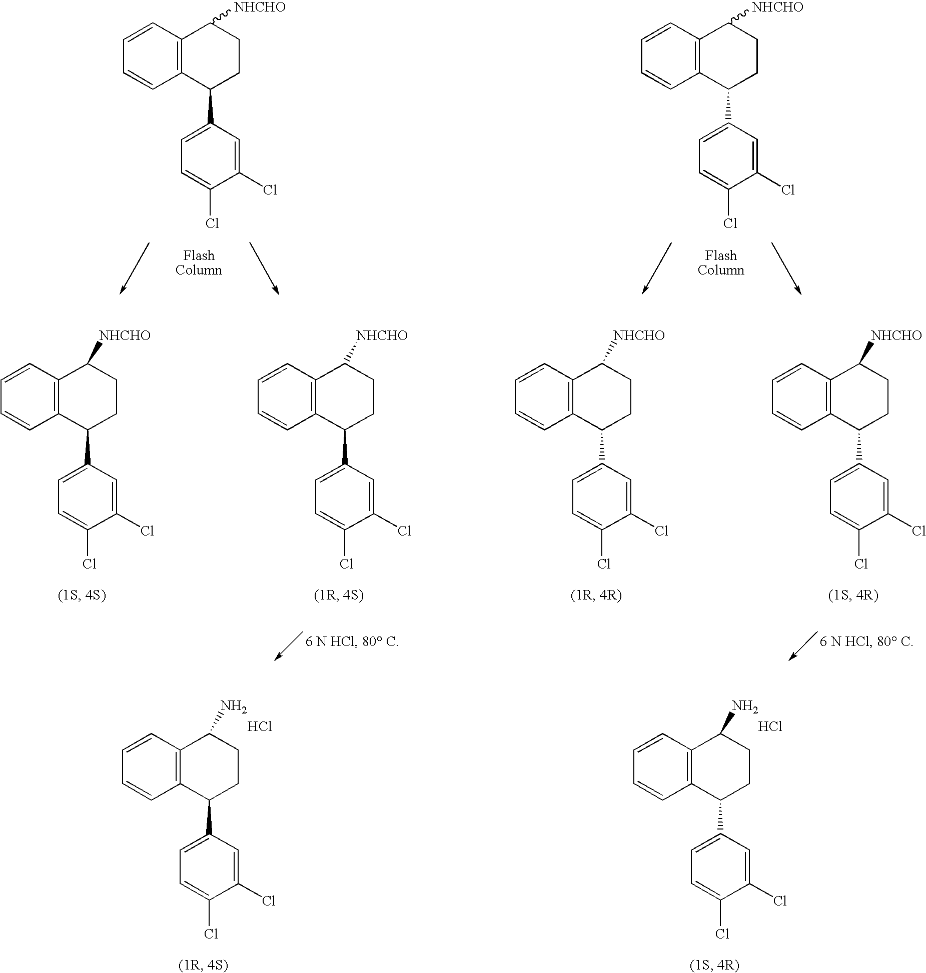

In the compound

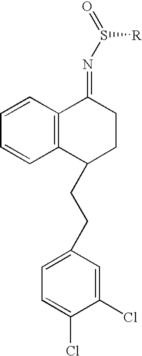

of Scheme 1,

### R is

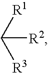

wherein R¹, R² and R³ are each independently alkyl. In a preferred embodiment of the compounds, R is tert-butyl.

N-[4-(3,4-dichlorophenyl)-1,2,3,4-tetrahydronaphthalen-1-yl]formamide, the intermediate in the synthesis shown in Scheme 1, exists in four stereoisomeric forms:

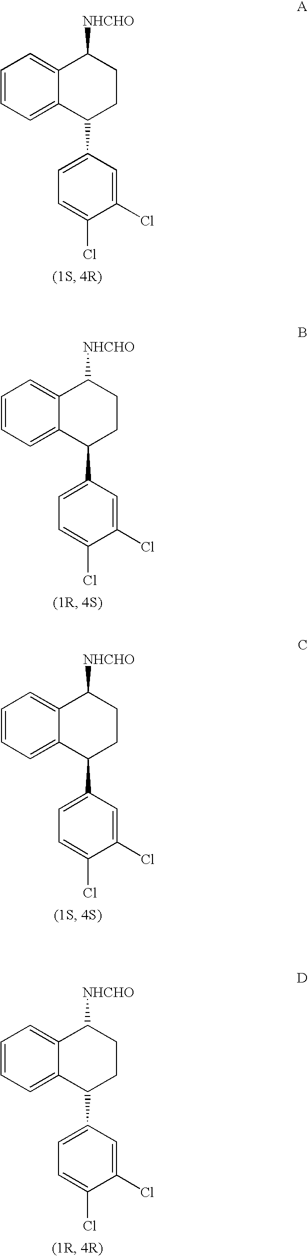

When N-[4-(3,4-dichlorophenyl)-1,2,3,4-tetrahydronaphthalen-1-yl]formamide is synthesized from achiral starting materials via non-stereoselective syntheses, all four isomers will be produced. The mixture can be readily separated into a racemic cis diastereomer and a racemic trans diastereomer by means, such as recrystallization or chromatography on achiral media, that rely on chemical and physical differences.

The trans diastereomer, represented as E below, is a 1:1 mixture of A and B. When E is hydrolyzed, PQ is produced, when A is hydrolyzed, P is produced, when B is hydrolyzed, Q is produced. The cis diastereomer, represented as F below, is a 1:1 mix of C and D.

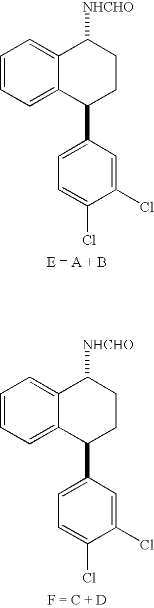

The graphic representations of racemic, ambiscalemic and scalemic or enantiomerically pure compounds used herein are taken from Maehr, J. Chem. Ed., 62:114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

Thus, formula PQ above indicates any mixture of the individual isomers P and Q, which share the trans relative configuration. Clearly, the most convenient mixture is the 1:1 racemate.

According to the present invention, a therapeutically effective amount of N-[4-(3,4-dichlorophenyl)-1,2,3,4-tetrahydronaphthalen-1-yl]formamide, which may be a pure isomer or a mixture of any or all of A, B, C and D, may also be administered to a person in need of therapy. Accordingly, the present invention encompasses a method for treating a pain disorder in a human, the method comprising administering to a person in need of treatment for a pain disorder, a therapeutically effective amount of a compound of formula K:

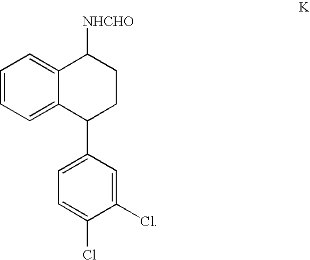

### The compound of formula K includes a compound of formula E:

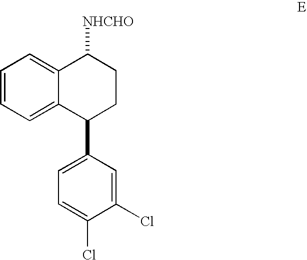

### The compound of formula K may also be a compound of formula F:

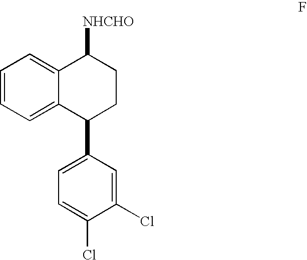

### The compound of formula K includes compounds of formulae A, B, C, or D:

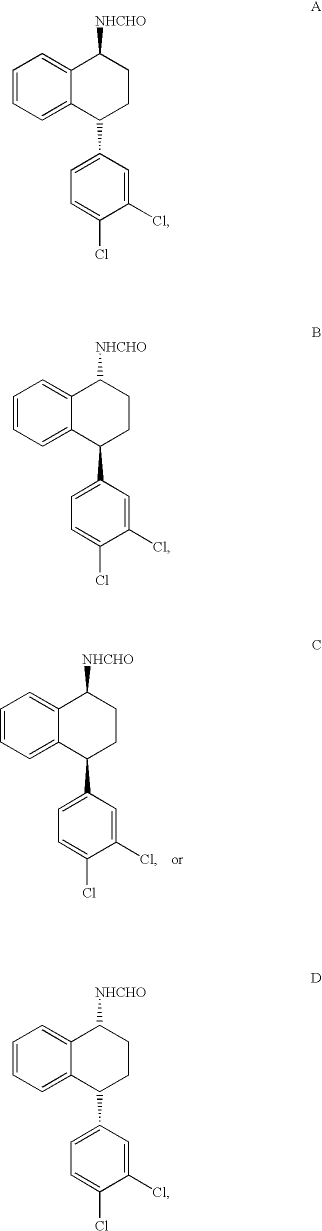

wherein A, B, C, or D is present in about 80% to about 100% enantiomeric excess. In one embodiment, A, B, C, or D is present in about 90% to about 100% enantiomeric excess. In another embodiment, A, B, C, or D is present in about 95% to about 100% enantiomeric excess. In yet another embodiment, A, B, C, or D is present in about 99% to about 100% enantiomeric excess.

In one embodiment, the compound of formula K is a mixture of A and B.

In another embodiment, the compound of formula K is a mixture of C and D.

Pain disorders treatable with the compounds of the invention include, but are not limited to neuropathic pain and fibromyalgia.

Neuropathic pain disorders treatable with the compounds of the invention include, but are not limited to: burning and tingling sensations, hypersensitivity to touch and cold, phantom limb pain, postherpetic neuralgia, diabetic peripheral neuropathy, and chronic pain syndrome. In one particular embodiment, chronic pain syndrome is reflex sympathetic dystrophy or causalgia.

Current approaches to treating neuropathic pain disorders in man are the selective inhibition of a single monoamine uptake mechanism or the dual inhibition of two of these molecular targets. Inhibition of neuronal uptake of all three of 5-HT, NE and DA using the methods of the present invention provides the clinician with the ability to treat the neuropathic pain disorders more effectively by elevating all of the monoamine levels in the nervous system simultaneously and over the same dose-range without the need to titrate separate drugs.

Fibromyalgia disorders treatable with the compounds of the invention include, but are not limited to: generalized fibromyalgia, primary fibromyalgia syndrome, secondary fibromyalgia syndrome, localized fibromyalgia, and myofascial pain syndrome.

Current approaches to treating fibromyalgia disorders in man are by increase of 5-HT and NE levels by use of reuptake inhibitors. Since the compounds of the present invention are potent 5-HT and NE reuptake inhibitors they are useful in increasing 5-HT and NE levels, which has a beneficial effect on symptoms associated with fibromyalgia disorders. Furthermore, the compounds of the present invention exhibit equipotent selective inhibition of neuronal uptake of an additional monoamine, DA. The capability to selectively elevate all of the monoamine levels simultaneously and over the same do se-range is an advantageous quality of the compounds used in the methods of the present invention.

Administration of compounds of the present invention results in a broad therapeutic profile. Due to the ability of the compounds of the present invention to inhibit monoamine uptake without affecting other receptors or ion channels, their administration can avoid or ameliorateside-effects that are associated with an Imbalance in the distribution of activity among 5-HT, NE and DA receptors. Such side effects may include extrapyramidal symptoms, elevated serum prolactin levels, sexual dysfunction (decreased libido, anorgasmia, ejaculatory dysfunction), breast pain, weight gain and insomnia.

The magnitude of a prophylacetic or therapeutic dose of a compound of formula A-F, P or Q will vary with the nature and severity of the condition to be treated and the route of administration. The dose will also vary according to the age, body weight and response of the individual patient. In general, the total daily dose ranges of compounds of the present invention will be from about 0.5 mg per day to about 100 mg per day, preferably about 1 mg per day to about 25 mg per day, in single or divided doses. It may be necessary to use dosages outside these ranges in some cases, as will be apparent to those in the art. Further, it is noted that the clinician or treating physician knows how and when to interrupt adjust or terminate therapy in conjunction with an individual patient's response.

Any suitable route of administration may be employed. For example, oral, rectal, intranasal, and parenteral (including trans- or subcutaneous, intramuscular, and intravenous) routes may be employed. Dosage forms can include tablets, troches, dispersions, suspensions, solutions, capsules and patches.

Pharmaceutical compositions of the present invention include, as active ingredient, a single compound, or a mixture of compounds, of formula A-F, P or Q, or a pharmaceutically acceptable salt of P or Q, together with a pharmaceutically acceptable carrier and, optionally, with other therapeutic ingredients.

Compositions suitable for oral, rectal, and parenteral administration are encompassed by the present invention. A preferred route of administration is oral. The compositions may be conveniently presented in unit dosage form and prepared by any of the methods well known in the art of pharmacy. Preferred unit dosage formulations are those containing a therapeutically effective dose, or an appropriate fraction thereof, of the active ingredient(s).

The compositions of the present invention will also include a pharmaceutically acceptable carrier. The carrier may take a wide variety of forms, depending on the route desired for administration, for example, oral or parenteral (including intravenous). In preparing the composition for oral dosage form, any of the usual pharmaceutical media may be employed, such as, water, glycols, oils, alcohols, flavoring agents, preservatives, and coloring agents in the case of oral liquid preparation, including suspension, elixirs and solutions. Carriers such as starches, sugars, microcrystalline cellulose, diluents, granulating agents, lubricants, binders and disintegrating agents may be used in the case of oral solid preparations such as powders, capsules and caplets, with the solid oral preparation being preferred over the liquid preparations. Preferred solid oral preparations are tablets or capsules, because of their ease of administration. If desired, tablets may be coated by a standard aqueous or nonaqueous techniques. Oral and parenteral sustained release dosage forms may also be used.

Oral syrups, as well as other oral liquid formulations, are well known to those skilled in the art, and general methods for preparing them are found in any standard pharmacy school textbook for example Remington: The Science and Practice of Pharmacy. Chapter 86 of the 19th edition of Remington entitled “Solutions, Emulsions, Suspensions and Extracts” describes in complete detail the preparation of syrups (pages 1503-1505) and other oral liquids.

Similarly, sustained release formulation is well known in the art, and Chapter 94 of the same reference, entitled “Sustained-Release Drug Delivery Systems,” describes the more common types of oral and parenteral sustained-release dosage forms (pages 1660-1675.) The relevant disclosure of each of these chapters is incorporated herein by reference. Because they reduce peak plasma concentrations, as compared to conventional oral dosage forms, controlled release dosage forms are particularly useful for providing therapeutic plasma concentrations while avoiding the side effects associated with high peak plasma concentrations that occur with conventional dosage forms.

### EXAMPLE 1

### Synthesis of Enantiomerically Enriched Compounds P and Q

### 1.1 Synthesis of 2-methyl-propane-2-sulfinic acid [4-(3,4-dichlorophenyl)-1,2,3,4-tetrahydro-naphthalen-y-yl]-amide (tetralone t-butanesulfinimine)

To a solution of 4-((3,4-dichlorophenyl)-3,4-dihydro-1-naphthalene (12 g) in THF (40 mL) was added (R)-t-butanesulfinamide (5.2 g) and Ti(OEt)₄ (85 mL 20%) in EtOH. The reaction mixture was heated to 60° C. for 13 h. The reaction mixture was cooled to rt, and poured into a brine solution (100 mL) with stirring. The suspension was then added to EtOAc (300 mL) and stirred for 10 min. The suspension was filtered and the filtrate was concentrated to ca 50 mL. One hundred milliliters of EtOAc was added and the organic phase was separated and concentrated to give a crude reaction mixture. The final products were isolated from the crude products by careful flash column chromatography using EtOAc and hexane (3:7 to 1:1) to give ca 3 g starting ketone, and (1R,4S)-4-(3,4-dichlorophenyl)-3,4-dihydro-1-naphthalenone tert-butanesulfinimine (2.5 g, first product) as an oil that solidified on standing. ¹H NMR (CDCl₃) δ 1.33 (S,9H), 2.10-2.20 (m, 1H), 2.28-2.38 (m, 1H) 2.88-2.98 (m, 1H), 3.34-3.44 (m 1H), 4.12-4.24 (m, 1H), 6.84-6.88 (m, 2H), 7.20 (s,1H), 7.25-7.40 (m, 3H), 8.22-8.28 (m, 1H). The other product (1R,4R)-4-(3,4-dichloro phenyl)-3-4-dihydro-1-naphthalenone tert-butanesulfinimine (3.0 g, second product, lower R_{f}) was isolated also as an oil that solidified on standing. ¹H NMR (CDCl₃) δ 1.34 (S, 9H), 2.05-2.18 (m, 1H), 2.28-2.38 (m, 1H), 3.15-3.25 (m, 2H), 4.16-4.22(m,1H), 6.84-6.88(m,2H), 7.20(s,1H), 7.25-7.40(m,3H), 8.22-8.28(m, 1H).

### 1.2 Synthesis of (R)-4-(3,4-dichlorophenyl)-3,4-dihydro-1-naphthalenone

(1R,4R)-4-(3,4-dichlorophenyl)3,4-dihydro-1-naphthalenone t-butanesulfinimine (3.0 g, second product in Example 1.1 above) was dissolved in MeOH (20 mL) and concentrated HCl (4 mL) at rt. The reaction mixture was stirred at rt to give a suspension. It was filtered and the solids were washed with hexane to give 1.2 g product. The enantiomeric purity was determined to be >99.3% by HPLC analysis with a ChiralPak AS 10:m, 4.6×250 mm, Hexane/IPA (90:10), TV 220 nm, R-isomer 8.23 min. S-isomer 12.25 min. ¹H NMR (CDCl₃) δ 2.20-2.32 (m, 1H), 2.42-2.53 (m, 1H) 2.57-2.78 (m,2H), 4.28 (dd=4.6, 8.1 Hz, 1H), 6.95 (dd, J=2.1, 7.6 Hz, 2H), 7.23 (d J=2.0 Hz, 1H), 7.37-50 (m, 3H), 8.13 (d, J=7.6 Hz, 1H). [α]=−66E (c=1, acetone).

### 1.3 Synthesis of (S)-4-(3,4-dichlorophenyl)-3,4-dihydro-1-naphthalenone

The previous procedure of Example 1.2 was used, except that the starting material was the (1R,4S)-4-(3,4-dichlorophenyl)-3,4-dihydro-1-naphthalenone tert-butanesulfinimine product of Example 1.1. 1.7 g of product (>99% ee) was obtained.

[α]=+62 (c=1, acetone). ¹H NMR spectrum of the product is the same as that of its enantiomer.

### 1.4 Synthesis of (1S,4R) and (1R,4R)-N-[4-(3,4-dichlorophenyl)-1,2,3,4-tetrahydro-naphthalen-1-yl]-formamide

(R)-4-(3,4-dichlorophenyl)-3,4-dihydro-1-naphthalenone (1.2 g) was added formic acid (3 mL) and formamide (3 mL). The reaction mixture was heated to 160-165° C. for 15 h under nitrogen atmosphere. The reaction mixture was cooled to room temperature and decanted the solvent. The residue solid was passed through flash column using EtOAc:Hexane (3:7 to 1:1) to give the (1R,4R)-formamide (400 mg, first spot), and the (1S,4R)-formamide (360 mg). ¹H NMR of the first product [(1R,4R)-isomer]: (CDCl₃) δ 1.80-2.10 (m, 3H), 2.10-2.20 (m, 1H), 4.00-4.10 (m, 1H), 5.22-5.30 (m, 1H), 6.10-6.20 (m, 1H), 6.80-6.90 (M, 1H), 6.90-6.96 (m, 1H), 7.10-7.40 (m, 5H), 8.22 (s, 1H). M+320. ¹H NMR of the second product [(1S,4R)-isomer: δ 1.64-1.90 (m, 2H), 2.10-2.28 (m, 2H), 4.10 (m, 1H), 5.38-5.42 (m, 1H), 5.82-6.05 (m, 1H), 6.80-6.90 (m, 2H), 7.10-40 (m, 5H), 8.28 (s, 1H). Mass Spec. M⁺ 320.

### 1.5 Synthesis of (1S,4R)-trans 4-(3,4-dichlorophenyl)-1,2,3,4-tetrahydro-1-napthalenamine HCl

(1S,4R) formamide (ca 300 mg) from Example 1.4 above was dissolved in MeOH (5 mL) followed by addition of 6N HCl (6 mL). The reaction mixture was heated to 80° C. for 2 h. The reaction mixture was cooled to rt for 1 h and filtered to collect the solid. It washed with acetone (3 mL) and dried to give the product (280 mg). Enantiomeric purity was determined to be >99.8% by HPLC analysis with a ChiralPak AD 10 μm, 4.6×250 mm, Hexane/IPA/DEA (99:1:0.1), UV 220 nm, (1R,4S)-isomer, 11.00 min. (1S,4R)-isomer 11.70 min [α]=−51° (C=1, MeOH). ¹H NMR (CD₃OD) δ 1.86-1.97 (m, 2H), 2.20-2.42 (m, 2H), 4.30 (broad s, 1H), 4.67 (broad s, 1H), 4.87 (s, 3H), 6.95-6.99 (m, 2H), 7.18 (s, 1H), 7.28-7.50 (m, m, 4H). M⁺ 293.

### 1.6 Synthesis of (1R,4S)-trans 4-(3,4-dichlorophenyl)-1,2,3,4-tetrahydro-1-napthalenamine HCl

(1R,4S)-trans 4-(3,4-dichlorophenyl)-1,2,3,4-tetrahydro-1-napthalenamine HCl was obtained similarly from (1R,4S) formamide with HCl hydrolysis. Ee of the product is >99.8% based on HPLC analysis with a ChiralPak AD 10:m, 4.6×250 mm, Hexane/IPA/DEA (99:1:0.1), UV 220 nm, (1R,4S)-isomer 11.00 min. (1S,4R)-isomer 11.70 min.

The experimental data presented below demonstrates that compound P and compound Q are potent inhibitors of the reuptake of NE, DA and 5-HT. The monoamine reuptake inhibitory properties of compounds P and Q make them useful in treatment of pain disorders and reduce side effects associated with imbalance in monoamine levels. The triple monoamine reuptake inhibition properties of compounds P and Q give them advantage in treatment of various pain disorders.

Furthermore, present studies demonstrated that compound P has potential for rapid onset of action and long duration of action. Compound P was shown to be a specific monoamine reuptake inhibitor with little additional pharmacologic activity. The lack of additional activity significantly improves its side effect profile over current compounds used in treatment of neuropathic pain, such as tricyclic antidepressants. Present experiments demonstrated no effect on ECG, heart rate, or blood pressure at doses up to those maximally tolerated in dogs. This shows an improved cardiovascular effect profile over other agents used to treat neuropathic pain.

### EXAMPLE 2

### Radioligand Binding Assays

Studies were performed to obtain and compare Kᵢ (inhibition constant) values of (1R,4S)-trans 4-(3,4-dichlorophenyl)-1,2,3,4-tetrahydro-1-napthalenamine (Compound P) and sertraline (ZOLOFT™ in radiolabeled binding assays.

The assays measured the affinity of the two compounds for transporters of the monoamines 5-HT, NE and DA. The assay for affinity to the 5-HT transporter was performed essentially as outlined in Tatsumi M. et al., Eur. J. Pharmacol. 340 249 (1997). The assay for affinity to the NE transporter was performed essentially as outlined in Pacholczyk T. et al., Nature, 350: 350 (1991). The assay for affinity to the DA transporter was performed essentially as outlined in Andersen P. H., J. Neurochem, 48: 1887 (1987). The transporters were human recombinant proteins expressed in mammalian cells. Binding to the 5-HT transporter was assessed by evaluating displacement of [³H]paroxetine (0.1 nM). Binding to the DA transporter was assessed by evaluating displacement of [³H]GBR 12935 (0.5 nM). Binding to the NE transporter was assessed by evaluating displacement of [³H]nisoxetine (0.3 nM). The results of the assays are presented in Table 1

TABLE 1
Kᵢ Values (nM) in Radioligand Binding Assays

| | Transporter | | |
|---|---|---|---|
| | Serotonin | Norepinephrine | Dopamine |
| | (5-HT) | (NE) | (DA) |
| Compound P | 23 | 20 | 16 |
| Sertraline (ZOLOFT ®) | <3.0 | 1,200 | 57 |

As could be seen in Table 1, (1R,4S)-trans 4-(3,4-dichlorophenyl)-1,2,3,4-tetrahydro-1-napthalenamme (Compound P) has high affinity to human recombinant 5-HT, NE and DA transporters. Unlike sertraline, a 5-HT specific reuptake inhibitor, Compound P has high and approximately equal affinity for all three transporters.

### EXAMPLE 3

### Functional Uptake Assays

(1R,4S)-trans 4-(3,4-dichlorophenyl)-1,2,3,4-tetrahydro-1-napthalenamine (Compound P), (1S,4R)-trans 4-(3,4-dichlorophenyl)-1,2,3,4-tetrahydro-1-napthalenamine (Compound Q), and a mixture of the two were tested for their ability to inhibit the functional uptake of radiolabeled 5-HT, NE, and DA into synaptosomes prepared from rat whole brain, hypothalamus, or corpus striatum, respectively. The 5-HT and NE assays were performed essentially as outlined in Perovic S. & Müller W. E. G., Arzneim.-Forsch. Drug Res., 45: 1145 (1995). The DA assay was performed essentially as outlined in Janowsky A. et al., Neurochem., 46: 1272 (1986).

IC₅₀ values (concentration inhibiting control activity by 50%) for Compounds P, Q, a mixture of the two, and some known monoamine reuptake inhibitors are shown in Table 2. Fluoxetine (PROZAC®) IC₅₀ values were taken from Wong et al., Neuropsychopharmacology, Jun, 8(4):337-44 (1993).

TABLE 2
IC₅₀ Values (nM) in Functional Uptake Assays

| | Serotonin | Norepinephrine | Dopamine |
|---|---|---|---|
| | (5-HT) | (NE) | (DA) |
| Compound P | 7.7 | 9.6 | 6.4 |
| Compound Q | 88 | 35 | 19 |
| 1:1 mixture of Compounds | 4.1 | 8.8 | 7.1 |
| P & Q | | | |
| Sertraline (ZOLOFT ®) | 1.6 | 310 | 48 |
| Fluoxetine (PROZAC ®) | 34 | 1,230 | 2,886 |
| Citalopram (CELEXA ™) | 3.2 | 7,900 | 17,000 |
| Venlafaxine (EFFEXOR ®) | 60 | 360 | 5,000 |
| Bupropion | NM | 611 | 294 |
| (WELLBUTRIN ®) | | | |

NM = not biologically meaningful

Unlike the 5-HT specific reuptake inhibitors (sertraline, fluoxetine and citalopram) and the “dual” reuptake inhibitors (venlafaxine and bupropion), Compounds P and Q potently inhibit the uptake of all three monoamines. Compound P is approximately equipotent in inhibiting the uptake of all three monoamines. Compound Q is approximately ten-fold less potent in inhibiting 5-HT reuptake, and three to four-fold less potent in inhibiting NE and DA reuptake when compared to Compound P.

### EXAMPLE 4

### Functional Uptake Assays of the Compounds of the Invention and Known Monoamine Reuptake Inhibitors using Recombinant Human Transporters

(1R,4S)-trans 4-(3,4-dichlorophenyl)-1,2,3,4-tetrahydro-1-napthalenamine (Compound P) and (1S,4R)-trans 4-(3,4-dichlorophenyl)-1,2,3,4-tetrahydro-1-napthalenamine (Compound Q) were also tested for their ability to inhibit functional uptake of radiolabeled 5-HT, NE, and DA into cells expressing recombinant human transporters. Cells expressing recombinant human transporters were obtained following the protocols of Gu H. et al., J. Biol. Chem., 269(10): 7124 (1994). Testing of the uptake inhibition was performed following protocols disclosed by Galli A. et al., J. Exp. Biol., 198: 2197 (1995), Giros B. et al., Mol. Pharmacol., 42: 383 (1992), Pristupa Z. B. et al., Mol. Pharmacol., 45: 125 (1994), and Gu H. et al., J. Biol. Chem., 269(10): 7124 (1994).

TABLE 3
IC₅₀ Values (nM) for inhibition of functional
uptake in human transporters

| | Serotonin | Norepinephrine | Dopamine |
|---|---|---|---|
| | (5-HT) | (NE) | (DA) |
| Compound P | 18.4 | 15.6 | 12.9 |
| Compound Q | 47.8 | 16.5 | 14.4 |
| Sertraline (ZOLOFT ®) | 1.2 | 718 | 158 |
| Venlafaxine (EFFEXOR ®) | 6.6 | 181 | 3,580 |
| Reference | 3.3 | 1.1 | 14.2 |
| | Fluoxetine | Desipramine | Nomifensine |

Consistent with the results obtained using rat brain as source material, both Compound P and Compound Q potently inhibited the functional uptake of 5-HT, NE, and DA.

### EXAMPLE 5

### In Vitro Receptor Binding Assays

Compound P and sertraline were compared in a broad in vitro screen of radioligand binding assays, which included 68 receptors. At a concentration of 10 μM, less than 50% inhibition of specific binding was observed in most of these receptors and the assays in which this was seen are listed in Table 4 below.

TABLE 4

| | | | | |
|---|---|---|---|---|
| Adenosine (A₁) | CGRP | GAL₁ | Neurokinin | TXA₂/PGH₂ |
| | | | (NK₂) | |
| Adenosine (A_{2A}) | CB₁ | GAL₂ | Neurokinin | P2X |
| | | | (NK₃) | |
| Adenosine (A₃) | CCK₁ | PDGF | Neuropeptide | P2Y |
| | | | Y (Y₁) | |
| α₁ adrenergic | CCK₂ | IL-8_{B} | Neuropeptide | 5-HT₆ |
| (nonselective) | | | Y (Y₂) | |
| α₂ adrenergic | Dopamine | TNF-α | NT₁ | 5-HT₇ |
| (nonselective) | (D₂) | | | |
| β₁ adrenergic | Dopamine | CCR₁ | δ (opioid) | γ (nonselective) |
| | (D₃) | | | |
| Angiotensin | Dopamine | Histamine | κ (opioid) | sst (nonselective) |
| (AT₁) | (D_{4.4}) | (H₁) | | |
| Angiotensin | Dopamine | MC₄ | μ (opioid) | VIP₁ |
| (AT₂) | (D₅) | | | |
| Benzodiazepine | Endothelin | ML₁ | ORL1 | V1a |
| (central) | (ET_{A}) | | | |
| Benzodiazepine | Endothelin | Muscreanic (M₄) | PACAP₁ | K⁺ Channel |
| (peripheral) | (ET_{B}) | | | |
| Bombesin | GABA | Neurokinin (NK₁) | PCP | Cl⁻ Channel |
| | (nonselective) | | | |

In thirteen assays, greater than 50% inhibition of specific binding was seen at a concentration of 10 μM. With the exception of the muscarinic (M₃) receptor, all Kᵢ values were 1 μM or higher and/or comparable to sertraline. The results are summarized in Table 5. The Kᵢ at M₃ is approximately 10-fold higher than Kᵢ values at the monoamine transporters (see Table 1) and 25 to 50-fold higher than effective concentrations in functional assays (see Table 2).

TABLE 5
Kᵢ Values (μM) for Compound P, sertraline and three reference compounds
in in vitro radioligand binding assays.

| | Compound P | Sertraline | Desipramine | Venlafaxine | Citalopram |
|---|---|---|---|---|---|
| B₂ | 1.5 | 4.5 | 1.5 | N.C. | >100 |
| H₂ | 4.0 | 4.5 | 5.2 | 89 | 8.9 |
| D₁ | 6.8 | 1.6 | 0.91 | N.C. | 3.1 |
| M₁ | 1.0 | 0.70 | 0.11 | >100 | 4.0 |
| M₂ | 5.0 | 3.7 | 0.83 | N.C. | 3.9 |
| M₃ | 0.17 | 1.8 | 0.07 | 33 | 3.9 |
| M₅ | 8.4 | 2.0 | 0.47 | >100 | 29 |
| 5-HT_{1A} | 1.1 | 2.4 | 7.0 | N.C. | 15 |
| 5-HT_{1B} | 1.0 | 8.5 | 2.7 | N.C. | N.C. |
| 5-HT_{2A} | 0.72 | 0.61 | 0.24 | >100 | 3.9 |
| 5-HT_{2C} | 5.1 | 5.6 | 0.43 | N.C. | 1.8 |
| Ca²⁺ Channel | 1.0 | 0.4 | 0.31 | 4.8 | 0.73 |
| Verapamil Site | | | | | |
| Na⁺ Channel Site 2 | 1.0 | 0.83 | 0.91 | 12 | 2.3 |

### EXAMPLE 6

### Assay of Inhibition of Carbachol-Induced Contraction of the Guinea Pig Ileum by Compound P and Sertraline

To determine whether Compound P is a muscarinic agonist or antagonist, its effects on the isolated Guinea pig ileum were evaluated and compared to those of sertraline. The assays were performed following protocols described by Clague et al., Brit. J. Pharmacol., 86: 163 (1985). To evaluate for possible agonist effects, graded concentrations of compound P or sertraline alone were tested. To evaluate for possible antagonist effects, the ability to antagonize carbachol-induced contraction was examined at different concentrations of test article (added before re-challenge with carbachol). The experiments demonstrated that Compound P is no an M₃ agonist. The results of experiments examining M₃ antagonism are presented in Table 6 below.

TABLE 6
IC₅₀ values for inhibition of carbachol-induced contraction
of the Guinea Pig ileum by Compound P and sertraline.

| Compound P | Sertraline |
|---|---|
| 9 μM | 7.6 μM |

Values are IC₅₀ (duplicate samples)
4-DAMP IC₅₀ = 33 nM;
pA₂ = 9.2

Both Compound P and sertraline inhibited carbachol induced contractions, and with approximately equal potency. The results demonstrate that, although Compound P has a higher affinity for the M₃ receptor, its functional M₃ antimuscrianic activity is comparable to that of sertraline.

### EXAMPLE 7

### In Vivo Pharmacologic Activity—Porsolt Forced Swim Test

Performance in the Porsolt forced swim test was evaluated for sertraline and Compound P following protocols outlined in Porsolt R. D., et al., Arch. Int. Pharmacodyn., 229: 327 (1997).

The Porsolt forced swim test is a model of “behavioral despair”, which has been used as an in vivo model for assessing potential antidepressant-like activity. Both test articles were dissolved in physiological saline (vehicle) and administered intraperitoneally. Doses for Compound P were 1.25, 2.5, and 5.0 mg/kg (N=3-6/dose/group). Doses for sertraline were 2.5, 5.0 and 10 mg/kg (N=2-6/group). Long-Evans derived rats were used. Table 7 shows results from the Porsolt swim test in which Compound P was administered twice daily starting 2 days prior to testing (for a total of 3 administrations) or 4 days prior to testing (for a total of 7 administrations).

TABLE 7
Porsolt Forced Swim Test in Rats

| | Dose | Mean Immobility Time (% of Total) ± SEM | | | |
|---|---|---|---|---|---|
| Treatment | (mg/kg) | N | Day 2 Study | N | Day 4 Study |
| Vehicle | — | 10 | 84 ± 2 | 5 | 77 ± 2 |
| Compound P | 1.25 | 6 | 77 ± 3 | 3 | 69 ± 4 |
| | 2.5 | 6 | 48 ± 6* | 6 | 46 ± 8* |
| | 5.0 | 6 | 49 ± 5* | 6 | 19 ± 6* |
| Sertraline | 2.5 | 6 | 86 ± 3 | 6 | 78 ± 3 |
| | 5.0 | 6 | 77 ± 2 | 6 | 64 ± 2* |
| | 10.0 | 6 | 72 ± 3* | 2 | 70 ± 4^{#} |

*Significantly different vs. vehicle by Dunnett's test (p < 0.05)
^{#}Error value is std dev since N = 2;

Compound P was active and significantly different than controls at 2.5 and 5.0 mg/kg (i.p.) in both the two-day and four-day Porsolt forced swim tests. It is noteworthy that a subchromic protocol, whereby animals receive a number of drug administrations over a period of at least 4-days, must be used to show antidepressant like activity for SSRIs (i.e. fluoxetine, as shown in Vazquez-Palacios et al. Pharmacol Biochem Behav 78: 165 (2004) and Lifschytz et al. Eur Neuropsychopharmacol 16: 115 (2006)), whereas atypical anti-depressants (i.e. desipramine, data not shown) demonstrate activity after only 3 drug administrations over 2-days. Therefore, the triple uptake activity of Compound P may provide features of both typical and atypical antidepressants, and may have a faster onset of action than the SSRIs. Although not a model of neuropathic pain, efficacy in this model demonstrates that Compound P crosses the blood-brain barrier and has the anticipated effects on monoamine reuptake in vivo. In addition, the rapid onset of the effects suggests that Compound P will exert beneficial effects semi-acutely.

### EXAMPLE 8

### In Vivo Pharmacologic Activity—Locomotor Activity Study

The effects on locomotor activity were studied in Long-Evans derived rats following protocol outlined in Dews P., Br. J. Pharmacol., 8: 46 (1953).

Table 8 shows the results from the locomotor activity study. Both sertraline and Compound P increased spontaneous locomotor activity above controls following a single intraperitoneal dose of 2.5 mg/kg (measurements initiated 10 minutes following dosing). This effect was still evident 2 hours following drug administration. On the basis of these studies, CNS effects and long duration of action may be anticipated at sufficient clinical doses.

TABLE 8
Total Activity Counts in a Spontaneous Locomotor Activity in Rats

| | Dose | | Total 1st | 71–80 | 91–100 | 111–120 | Total 2^{nd} |
|---|---|---|---|---|---|---|---|
| | (mg/kg) | N | Hour | Minutes | Minutes | Minutes | Hour |
| Vehicle | 0 | 10 | 396 ± 36 | 12 ± 4 | 12 ± 8 | 9 ± 4 | 68 ± 28 |
| Compound P | 2.5 | 6 | 577 ± 54¹ | 49 ± 6 | 54 ± 9 | 34 ± 9 | 257 ± 36* |
| | 5.0 | 6 | 444 ± 91 | 38 ± 3 | 26 ± 26 | 23 ± 14 | 160 ± 32 |
| Sertraline | 2.5 | 6 | 559 ± 57² | 34 ± 9 | 25 ± 9 | 30 ± 10 | 211 ± 60* |
| | 5.0 | 6 | 327 ± 55 | 32 ± 11 | 14 ± 9 | 16 ± 11 | 95 ± 41 |

Values shown are mean ± SEM.
*Significantly different vs. vehicle by one way ANOVA followed by post-hoc tests (p < 0.05);
¹p < 0.056;
²p < 0.085 vs. vehicle.

### EXAMPLE 9

### In Vitro Pharmacologic Activity Study—hERG Channel Current Study

Studies were conducted to determine effects on I_{Kr}, the rapidly activating delayed rectifier cardiac potassium current in the human heart. In order to determine the effects of Compound P on I_{Kr}, its effect on HERG channel current in stably transfected HEK293 cells was evaluated. In this assay system, HERG channel current serves as a surrogate for I_{Kr}. The results for Compound P are summarized and compared to those of known antidepressants in Table 9 below. Results for citalopram are from Witchel et al., FEBS Lett., 512(1-3): 59 (2002).

TABLE 9
Effects of Sertraline, Fluoxetine, Citalopram and Compound P
on hERG Channel Current

| Compound | IC₅₀ (μM) |
|---|---|
| Compound P | 0.8 |
| Sertraline | 1.1 |
| Fluoxetine | 0.5 |
| Citalopram | 4.0 |

In studies evaluating the effect of Compound P on hERG channel current in stably transfected BEK293 cells, Compound P was found to inhibit hERG channel current in a concentration-dependent manner, with an IC₅₀ between 0.7 and 0.8 μM. Sertraline, as a comparator control, also inhibited hERG channel current, with an IC₅₀ of 1.1 μM. Their effects on HERG channel current are similar to those of fluoxetine and citalopram.

### EXAMPLE 10

### In Vivo Pharmacologic Activity Study—Cardiovascular Safety Study

A cardiovascular safety study was conducted with Compound P in conscious dogs. The purpose was to assess any effects of Compound P on heart rate, blood pressure and ECG parameters at tolerated doses, and compare any effects to those of sertraline.

Conscious, telemetered Beagle dogs were orally administered single doses of either Compound P or sertraline at 0.5, 1.5 and 5 mg/kg. There were four animals/compound and each animal within the treatment group was exposed to the three doses (plus a empty capsule control), in a dose ramp-up manner with a minimum of 72 hours between doses.

Both Compound P and sertraline had no effect on heart rate, blood pressure or ECG parameters (including QTc) at the doses evaluated.

These results demonstrate that Compound P's effects on HERG channel current (a surrogate for I_{Kr}) did not translate into an effect on QTc in vivo at doses up to those maximally tolerated in dogs, and that it does not have effects on heart rate or blood pressure, issues shared by the tricyclic antidepressants.

### EXAMPLE 11

### In Vivo Testing in an SNL Model of Neuropathic Pain

The Spinal Nerve Ligation (SNL) model [Kim and Chung, Pain 50, 355-363 (1992)] was used to induce chronic neuropathic pain. The animals were anesthetized with isoflurane, the left L5 transverse process was removed, and the L5 and L6 spinal nerves were tightly ligated with 6-0 silk suture. The wound was then closed with internal sutures and external staples. Wound clips were removed 10-11 days following surgery.

Mechanical allodynia testing: Baseline, post-injury and posttreatment values for non-noxious mechanical sensitivity were evaluated using 8 Semmes-Weinstein filaments (Stoelting, Wood Dale, Ill., USA) with varying stiffness (0.4, 0.7, 1.2, 2.0, 3.6, 5.5, 8.5, and 15 g) according to the up-down method (Chaplan et al., 1994). Animals were placed on a perforated metallic platform and allowed to acclimate to their surroundings for a minimum of 30 minutes before testing. The mean and standard error of the mean (SEM) were determined for each animal in each treatment group. Since this stimulus is normally not considered painful, significant injury-induced increases in responsiveness in this test are interpreted as a measure of mechanical allodynia. Mechanical allodynia was evaluated 14 days post-surgery.

Mechanical hyperalgesia testing: Baseline, post-injury and post-treatment values for non-noxious mechanical sensitivity were evaluated using a Paw Pressure Analgesymeter (7200, Ugo Basile, Comerio, Italy) which generates a linearly increasing mechanical force. The mechanical nociceptive stimulus was applied to the plantar surface of the hind paws by a dome-shaped plastic tip placed between the 3^{rd} and 4^{th} metatarsus. To avoid tissue damage, a cutoff pressure was set at 390 g. Mechanical thresholds were defined as the force in grams at the first pain behavior, which includes paw withdrawal, struggle and/or vocalization. Paw pressure test was performed on each hind paw for each animal. The mean and standard error of the mean (SEM) were determined for each paw, in each treatment group. Mechanical hyperalgesia was evaluated 24 days post-surgery.

Ten rats were studied per group and the tests were performed blind. (1R,4S)-4-(3,4-dichlorophenyl)-1,2,3,4-tetrahydro-1-naphthalenamine (P) was evaluated at 3 doses (5, 10 and 15 mg/kg), administered orally and compared with a vehicle control group. Gabapentin (100 mg/kg) and duloxetine (30 mg/kg) administered orally under the same experimental conditions, were used as comparator substances. Mechanical allodynia and hyperalgesia testing were conducted at baseline and 1, 2 and 4 hours after test article administration.

Results: The results for P on mechanical allodynia and mechanical hyperalgesia are summarized in Tables 10 and 11, respectively. The mechanical allodynia results showed that P reduced allodynia at the highest dose level tested (15 mg/kg) during the 4 hour test period. Efficacy of P appeared to be similar to gabapentin. Duloxetine showed modest anti-allodynic effects; however, effects were not statistically significant. The mechanical hyperalgesia results showed that P significantly reduced mechanical hyperalgesia at all dose levels. Gabapentin and Duloxetine also produced robust and sustained anti-hyperalgesic effects at all time points tested.

TABLE 10
Effects of P, Duloxetine and Gabapentin on Mechanical
Allodynia (i.e., Mean ± SEM Paw Withdrawal
Thresholds) in the Rat SNL Model.

| | Testing Time | | | |
|---|---|---|---|---|
| | (Hours after Test Article Administration) | | | |
| Treatment | 0 (Baseline) | 1 | 2 | 4 |

Vehicle (PO)

| | | | | |
|---|---|---|---|---|
| 0 | 2.9 ± 0.2 | 5.1 ± 0.3 | 5.4 ± 0.5 | 3.8 ± 0.4 |

P (mg/kg, PO)

| | | | | |
|---|---|---|---|---|
| 5 | 3.1 ± 0.2 | 3.7 ± 0.3 | 3.2 ± 0.2 | 3.9 ± 0.2 |
| 10 | 2.8 ± 0.2 | 5.0 ± 0.3 | 5.7 ± 0.5 | 6.6 ± 0.4 |
| 15 | 2.9 ± 0.1 | 8.8 ± 0.6 | 7.5 ± 0.5 | 13.6 ± 0.3* |

Duloxetine (mg/kg, PO)

| | | | | |
|---|---|---|---|---|
| 30 | 2.6 ± 0.2 | 7.0 ± 0.5 | 7.9 ± 0.6 | 8.1 ± 0.6 |

Gabapentin (mg/kg, PO)

| | | | | |
|---|---|---|---|---|
| 100 | 2.7 ± 0.1 | 6.6 ± 0.4 | 9.8 ± 0.5* | 6.2 ± 0.4 |

ANOVA: * p < 0.05

TABLE 11
Effects of P, Duloxetine and Gabapentin on Mechanical Hyperalgesia
(i.e., Mean ± SEM Paw Pressure Thresholds) in the Rat SNL Model.

| | Testing Time | | | |
|---|---|---|---|---|
| | (Hours after Test Article Administration) | | | |
| Treatment | 0 (Baseline) | 1 | 2 | 4 |

Vehicle (PO)

| | | | | |
|---|---|---|---|---|
| 0 | 125 ± 17 | 101 ± 15 | 104 ± 35 | 99 ± 26 |

P (mg/kg, PO)

| | | | | |
|---|---|---|---|---|
| 5 | 147 ± 17 | 183 ± 35 | 240 ± 32 | 257 ± 37* |
| 10 | 152 ± 18 | 213 ± 32* | 309 ± 27* | 282 ± 30* |
| 15 | 164 ± 22 | 281 ± 37* | 251 ± 37* | 269 ± 37* |

Duloxetine (mg/kg, PO)

| | | | | |
|---|---|---|---|---|
| 30 | 153 ± 9 | 306 ± 30* | 345 ± 22* | 330 ± 25* |

Gabapentin (mg/kg, PO)

| | | | | |
|---|---|---|---|---|
| 100 | 135 ± 20 | 219 ± 32* | 236 ± 29* | 255 ± 29* |

### EXAMPLE 12

### In Vivo Testing in a Model of Persistent Pain—Formalin Paw Test in the Rat (Late Phase)

Additional studies were conducted to test Compound P (same doses as those of the above described SNL model of neuropathic pain study) in a formalin model of persistent pain. The method, which detects analgesic/anti-inflammatory activity, followed that described by Wheeler-Aceto et al. (Psychopharmacology, 104, 35-44, 1991). Rats were given an intraplantar injection of 5% formalin (50 μl) into the posterior left paw. This treatment induced a recognizable flinching response in control animals. The number of flinches was counted for 15 minutes, beginning 20 minutes after injection of formalin. Eight rats were studied per group. The test was performed blind. Compound P was evaluated at 3 doses (5, 10 and 15 mg/kg), administered orally 60 minutes before the test (i.e. 40 minutes before formalin), and compared with a vehicle control group. Morphine (128 mg/kg) and duloxetine (30 mg/kg) administered orally under the same experimental conditions, were used as comparator substances. Data were analyzed by comparing treated groups with vehicle control using unpaired Mann-Whitney U tests.

Results The results for P are summarized in Table 12. In summary, P dose-dependently reduced the number of flinches observed during the late phase (20-25 minutes after formalin injection) of the formalin test. Morphine also significantly reduced flinching behavior; however, duloxetine had no effect under the testing conditions.

TABLE 12
Effects of P and morphine on flinching behavior in the late
phase of the formalin flinch test in rats.

| TREATMENT | NUMBER OF FLINCHES | | |
|---|---|---|---|
| (mg/kg) | (20 to 35 min after formalin) | | |
| p.o. 60 min before the test | | p | % change |
| (i.e. 40 min before formalin) | mean ± s.e.m. | value | from control |
| Vehicle | 89.4 ± 14.2 | — | — |
| P (5) | 89.0 ± 18.4NS | 0.8336 | 0% |
| P (10) | 76.4 ± 15.2NS | 0.5632 | −15% |
| P (15) | 44.6 ± 13.6* | 0.0357 | −50% |
| Duloxetine (30) | 71.4 ± 17.2NS | 0.3717 | −20% |
| Morphine (128) | 0.0 ± 0.0*** | 0.0003 | −100% |

Mann-Whitney U test:
NS = Not Significant;
*= p < 0.05;
***= p < 0.001

### Test/control articles administered 60 minutes before formalin injection (30 minutes for Group 5).

In a second test, flinching behaviors were measured for 90 minutes after formalin injection using an automated detection system. Animals were tested for paw movement responses to injection of a 5% formalin solution (50 μl in saline) using the Automated Nociception Analyzer (Yaksh et al. J Appl Physiol 90:2386-402, 2001). This device used a magnetic detection system to measure paw movements, called “flinches”. Small metal bands were attached to the left hind paw of rats just before placement into individual circular test chambers 30 minutes prior to formalin injection. Rats were injected with P (5, 10 and 15 mg/kg,), duloxetine (30 mg/kg) or vehicle 60 minutes prior to formalin injection. In addition to duloxetine, morphine (6 mg/kg, subcutaneously) was used as a comparator substance. Animals were treated with morphine 30 minutes prior to the formalin injection. To initiate the experiment, the rats were injected with formalin subcutaneously on the dorsal surface of the left hind paw and placed in the test chambers. The instrument subsequently recorded rapid foot movements counted in one minute epochs.

The results for P in this second test are summarized in Table 13. In summary, P appeared to dose-dependently increase the number of flinches observed during the late phase (10-60 minutes after formalin injection) of the formalin test. Morphine significantly reduced flinching behavior; however, duloxetine had no effect on late phase flinching behavior. P and duloxetine had no effect on the acute phase of the formalin test (0-10 minutes after formalin injection). In contrast, morphine significantly reduced acute phase flinching behavior.

TABLE 13
Effects of P, duloxetine and morphine on flinching behavior
(Mean ± SEM) in the acute and late phase of the formalin test in rats.

Number of Flinches

| | | |
|---|---|---|
| | Acute | Late |
| Treatment | Phase | Phase |

Vehicle (PO)

| | | |
|---|---|---|
| 0 | 187 ± 30 | 1041 ± 156 |

P (mg/kg, PO)

| | | |
|---|---|---|
| 5 | 194 ± 33 | 1263 ± 181 |
| 10 | 143 ± 27 | 1679 ± 163* |
| 15 | 212 ± 40 | 2236 ± 153* |

Duloxetine (mg/kg, PO)

| | | |
|---|---|---|
| 30 | 168 ± 24 | 803 ± 112 |

Morphine (mg/kg, SC)

| | | |
|---|---|---|
| 100 | 36 ± 9* | 32 ± 13* |

*p < 0.05

Conclusion for formalin tests: While P dose-dependently reduced flinching behavior in one experiment, dose-dependent increases in flinching were observed in a second experiment. The reason for the discrepancy is likely related to the different scoring techniques used in each experiment. In the study that observed increases in flinching behavior, P dose-dependently increased baseline locomotor activity. Furthermore, an automated flinching apparatus was used to record flinching and the increases in flinching responses appeared to be correlated with the dose-dependent increases in locomotor activity. The automated detection apparatus is not able to distinguish between flinching behavior and overall increased locomotor activity. In the experiment where P exhibited a dose-dependent suppression of late phase formalin flinch behavior, a manual scoring technique was used to record flinching. Taken together, the findings support efficacy of P in the formalin flinch test.

### EXAMPLE 13

### Formulations

Compound P (or other compound of the invention) and silicon dioxide are dry mixed, the first portion of croscarmellose is added and the mixture is further dry mixed. The magnesium stearate is added, dry mixed and the mixture is run through a roller compactor and mill. The resulting dry granulate is mixed with the remaining three ingredients and compressed into tablets.

Powder-filled Capsules - Composition per unit dosage

| | | |
|---|---|---|
| Compound P | 5 | mg |
| Lactose | 250 | mg |
| Corn starch | 60 | mg |
| Magnesium stearate | 5 | mg |
| Total | 320 | mg |

Compound P, lactose and cornstarch, in the proportions shown above, are blended until uniform and then the magnesium stearate is blended into the resulting powder, which is sieved and filled into suitably sized, two-piece, hard gelatin capsules using conventional machinery. Other doses may be prepared by altering the fill weight and, if necessary, changing the capsule size to suit.

Pharmaceutical formulations of the formamides A-F may be prepared in similar fashion.

The present invention has been described with regard to preferred embodiments. However, it will be obvious to persons skilled in the art that a number of variations and modifications can be made without departing from the scope of the invention as described in the following claims.

All patents, publications, or other references that are listed herein are hereby incorporated by reference.

1. A method for treating a pain disorder in a human, the method comprising administering to a person in need of therapy for a pain disorder a therapeutically effective amount of a compound chosen from (1R,4S)-4-(3,4-dichlorophenyl)-1,2,3,4-tetrahydro-1-naphthalenamine (P); (1S,4R)-4-(3,4-dichlorophenyl)-1,2,3,4-tetrahydro-1-naphthalenamine (Q); 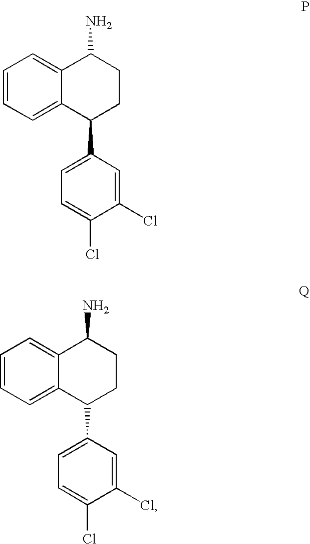
mixtures of P and Q; and pharmaceutically acceptable salts thereof.

2. A method according to claim 1, wherein the pain disorder is a neuropathic pain disorder.

3. A method according to claim 1, wherein the pain disorder is fibromyalgia.

4. A method according to claim 1, wherein the compound is (1R,4S)-4-(3,4-dichlorophenyl)-1,2,3,4-tetrahydro-1-naphthalenamine (P) or a pharmaceutically acceptable salt thereof.

5. A method according to claim 1, wherein the compound is (1S,4R)-4-(3,4-dichlorophenyl)-1,2,3,4-tetrahydro-1-naphthalenamine (Q) or a pharmaceutically acceptable salt thereof.

6. A method according to claim 1, wherein the compound is a mixture of (1R,4S)-4-(3,4-dichlorophenyl)-1,2,3,4-tetrahydro-1-naphthalenamine (P) and (1S,4R)-4-(3,4-dichlorophenyl)-1,2,3,4-tetrahydro-1-naphthalenamine (Q) or a pharmaceutically acceptable salt thereof.

7. A method according to claim 1, wherein the compound is administered in a pharmaceutical composition, wherein the pharmaceutical composition comprises the compound or a pharmaceutically acceptable salt thereof and a pharmaceutically acceptable carrier.

8. A method according to claim 7, wherein the pharmaceutical composition is administered in the form of a tablet or a capsule.

9. A method according to claim 2, wherein the neuropathic pain disorder is chosen from burning sensations, tingling sensations, hypersensitivity to touch, hypersensitivity to cold, phantom limb pain, postherpetic neuralgia, diabetic peripheral neuropathy, and chronic pain syndrome.

10. A method according to claim 9, wherein the chronic pain syndrome is chosen from reflex sympathetic dystrophy and causalgia.

11. A method according to claim 3, wherein the fibromyalgia disorder is chosen from generalized fibromyalgia, primary fibromyalgia syndrome, secondary fibromyalgia syndrome, localized fibromyalgia, and myofascial pain syndrome.

12. A method for treating a pain disorder in a human, the method comprising administering to a person in need of therapy for a pain disorder a therapeutically effective amount of a compound chosen from (1R,4S)-4-(3,4-dichlorophenyl)-1,2,3,4-tetrahydro-1-naphthalenamine (P); (1S,4R)-4-(3,4-dichlorophenyl)-1,2,3,4-tetrahydro-1-naphthalenamine (Q); 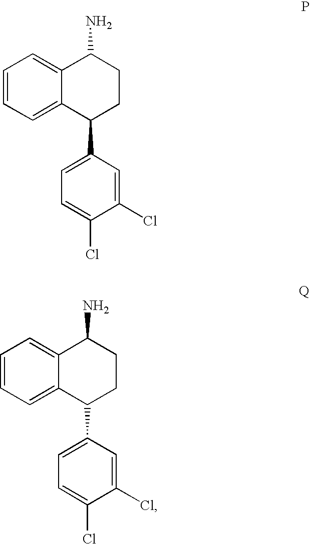
and pharmaceutically acceptable salts thereof; wherein P or Q is present in about 80% to about 100% enantiomeric excess.

13. A method according to claim 12, wherein the pain disorder is a neuropathic pain disorder.

14. A method according to claim 12, wherein the pain disorder is fibromyalgia.

15. A method according to claim 12, wherein P or Q is present in about 90% to about 100% enantiomeric excess.

16. A method according to claim 12, wherein P or Q is present in about 95% to about 100% enantiomeric excess.

17. A method according to claim 12, wherein P or Q is present in about 99% to about 100% enantiomeric excess.

18. A method according to claim 12, wherein the compound is (1R,4S)-4-(3,4-dichlorophenyl)-1,2,3,4-tetrahydro-1-naphthalenamine (P) or a pharmaceutically acceptable salt thereof.

19. A method according to claim 12, wherein the compound is (1S,4R)-4-(3,4-dichlorophenyl)-1,2,3,4-tetrahydro-1-naphthalenamine (Q) or a pharmaceutically acceptable salt thereof.

20. A method according to claim 12, wherein the compound is administered in a pharmaceutical composition, wherein the pharmaceutical composition comprises the compound or a pharmaceutically acceptable salt thereof and a pharmaceutically acceptable carrier.

21. A method according to claim 20, wherein the pharmaceutical composition is administered in the form of a tablet or a capsule.

22. A method according to claim 13, wherein the neuropathic pain disorder is chosen from burning sensations, tingling sensations, hypersensitivity to touch, hypersensitivity to cold, phantom limb pain, postherpetic neuralgia, diabetic peripheral neuropathy, and chronic pain syndrome.

23. A method according to claim 22, wherein the chronic pain syndrome is chosen from reflex sympathetic dystrophy and causalgia.

24. A method according to claim 14, wherein the fibromyalgia disorder is chosen from generalized fibromyalgia, primary fibromyalgia syndrome, secondary fibromyalgia syndrome, localized fibromyalgia, and myofascial pain syndrome.

25. A method for treating a pain disorder in a human, the method comprising administering to a person in need of treatment for a pain disorder, a therapeutically effective amount of a compound of formula K:

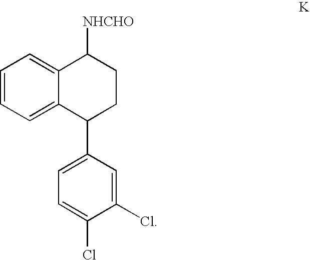

26. A method according to claim 25, wherein the pain disorder is a neuropathic pain disorder.

27. A method according to claim 25, wherein the pain disorder is fibromyalgia.

28. A method according to claim 25, wherein the compound of formula K is a compound of formula E:

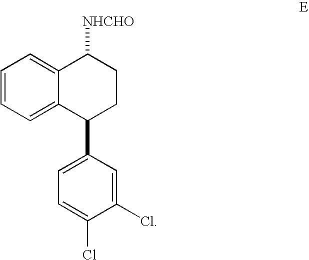

29. A method according to claim 25, wherein the compound of formula K is a compound of formula F:

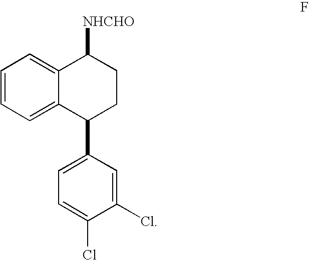

30. A method according to claim 25, wherein the compound of formula K is a compound of formula A, B, C, or D: 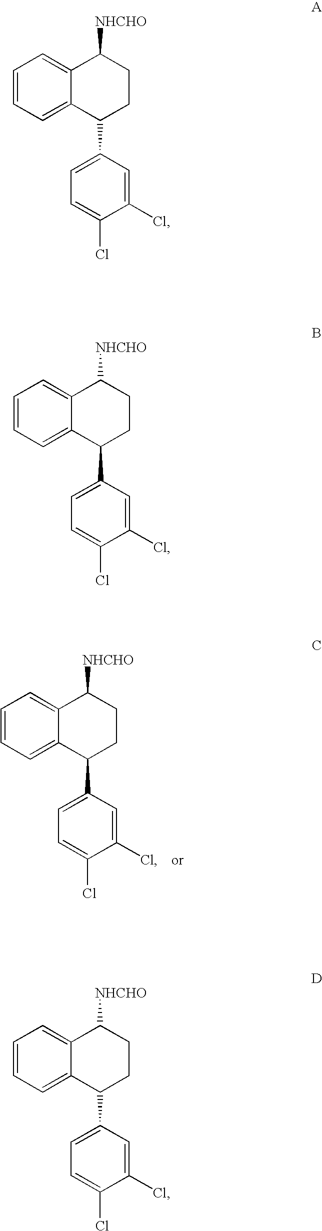
wherein A, B, C, or D is present in about 80% to about 100% enantiomeric excess.

31. A method according to claim 30, wherein A, B, C, or D is present in about 90% to about 100% enantiomeric excess.

32. A method according to claim 30, wherein A, B, C, or D is present in about 95% to about 100% enantiomeric excess.

33. A method according to claim 30, wherein A, B, C, or D is present in about 99% to 100% enantiomeric excess.

34. A method according to claim 25, wherein the compound of formula K is a mixture of (1S,4R)-N-[4-(3,4-dichlorophenyl)-1,2,3,4-tetrahydronaphthalen-1-yl]formamide (A) and (1R,4S)-N-[4-(3,4-dichlorophenyl)-1,2,3,4-tetrahydronaphthalen-1-yl]formamide (B).

35. A method according to claim 25, wherein the compound of formula K is a mixture of (1S,4S)-N-[4-(3,4-dichlorophenyl)-1,2,3,4-tetrahydronaphthalen-1-yl]formamide (C) and (1R,4R)-N-[4-(3,4-dichlorophenyl)-1,2,3,4-tetrahydronaphthalen-1-yl]formamide (D).

36. A method according to claim 25, wherein the compound of formula K is administered in a pharmaceutical composition, wherein the pharmaceutical composition comprises the compound of formula K and a pharmaceutically acceptable carrier.

37. A method according to claim 36, wherein the pharmaceutical composition is administered in the form of a tablet or a capsule.

38. A method according to claim 26 wherein the neuropathic pain disorder is chosen from burning sensations, tingling sensations, hypersensitivity to touch, hypersensitivity to cold, phantom limb pain, postherpetic neuralgia, diabetic peripheral neuropathy, and chronic pain syndrome.

39. A method according to claim 38 wherein the chronic pain syndrome is chosen from reflex sympathetic dystrophy and causalgia.

40. A method according to claim 27 wherein the fibromyalgia disorder is chosen from generalized fibromyalgia, primary fibromyalgia syndrome, secondary fibromyalgia syndrome, localized fibromyalgia, and myofascial pain syndrome.
